(19) United States
(12) Patent Application Publication
(10) Pub. No.: US 2020/0060638 A1
(43) Pub. Date: Feb. 27, 2020

(54) APPARATUS FOR X-RAY BONE DENSITOMETRY

(71) Applicant: EUROTEC MEDICAL SYSTEMS S.R.L., Scanzorosciate, Bergamo (IT)

(72) Inventor: Giovanni Fiorenzo ASSOLARI, Torre De' Roveri, Bergamo (IT)

(21) Appl. No.: 16/346,829

(22) PCT Filed: Oct. 31, 2017
(86) PCT No.: PCT/IB2017/056749

§ 371 Date: May 1, 2019

(30) Foreign Application Priority Data
Nov. 2, 2016 (IT) 102016000109999

(51) Int. Cl. A61B 6/00; A61B 6/04; A61B 6/06

(52) U.S. Cl. CPC A61B 6/505; A61B 6/0407; A61B 6/4441; A61B 6/482; A61B 6/4035; A61B 6/4476; A61B 6/06

(57) ABSTRACT

An apparatus for bone densitometry (1) includes a patient-carrier frame (2) fitted with a support surface (21) for a patient and an arm (3) movable with respect to the frame (2) in a longitudinal direction (X) from the feet to the head of the patient. The arm is fitted with a generator-detector assembly having an X-ray generator (31) and an X-ray detector (32), mechanically aligned so that the collimated beam (F) of X-rays produced by the generator (31) centers the detector (32). In particular, the frame is a monocoque (2) and fully supports, at the support surface (21), the arm (3) in order to leave the space underneath the frame (2) free. Advantageously, moreover, in the case of apparatus with frame (2) made of aluminum, the frame itself also serves as a filter for X-rays.

The present invention relates to an apparatus for bone densitometry, in particular, an apparatus for bone densitometry which uses a DEXA method.

The analysis of bone densitometry allows the determination of bone density in the districts at greater risk of fracture (such as the spine, femur and forearm, as well as the whole body) and supports the physician in diagnosing osteoporosis, in fracture risk assessment and in monitoring the response to therapy.

One of the most accurate and reliable techniques for the assessment of bone densitometry, with insignificant radiation doses to the patient, is DEXA (Dual Energy X-Ray Absorptiometry).

This method is based on the assessment of bone mineral density (BMD) and provides two very important indicators: the T-Score (deviation of the mineral density compared to the normal value at about 30 years age and the Z-Score (deviation of the mineral density compared to subjects of the same age). The examinations carried out by the machine also allow assessing the body composition (lean mass, fat mass and bone mass) in certain clinical conditions and setting and monitoring the most suitable dietary treatments.

Apparatuses for bone densitometry are known in the field; an example is described in document EP0761166. This known apparatus comprises a patient-carrier table and a movable arm provided with an RX generator-detector assembly. The arm is movable longitudinally by means of a movement system (guide rails and belts) resting directly on the ground. Therefore, the space below the patient-carrier table is completely occupied by the movement system of the movable arm, making it difficult to clean the area below it.

Moreover, this known apparatus comprises an extremely large number of components, which makes the assembly, installation and maintenance of the apparatus complex.

The object of the present invention is to propose an apparatus for bone densitometry with DEXA method which allows obviating the drawbacks mentioned above, in particular that has a very simple structure that allows having free space below the patient-carrier table, while being able to withstand high weight and height patients.

Said objects are achieved with an apparatus for bone densitometry according to the following claims.

The details and the advantages of the apparatus for bone densitometry according to the present invention will be apparent from the following description of preferred embodiments thereof, provided purely by way of a non-limiting example, with reference to the accompanying drawings, in which:

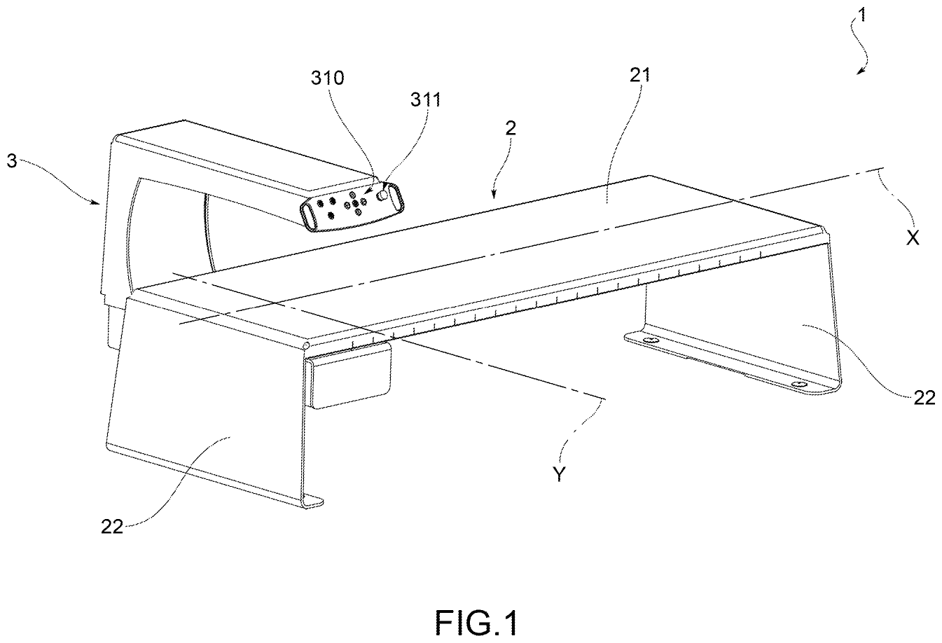

FIG. 1 is an overall view of an apparatus for bone densitometry according to the invention, fitted with a patient-carrier frame and a movable arm fitted with an RX generator-detector assembly;

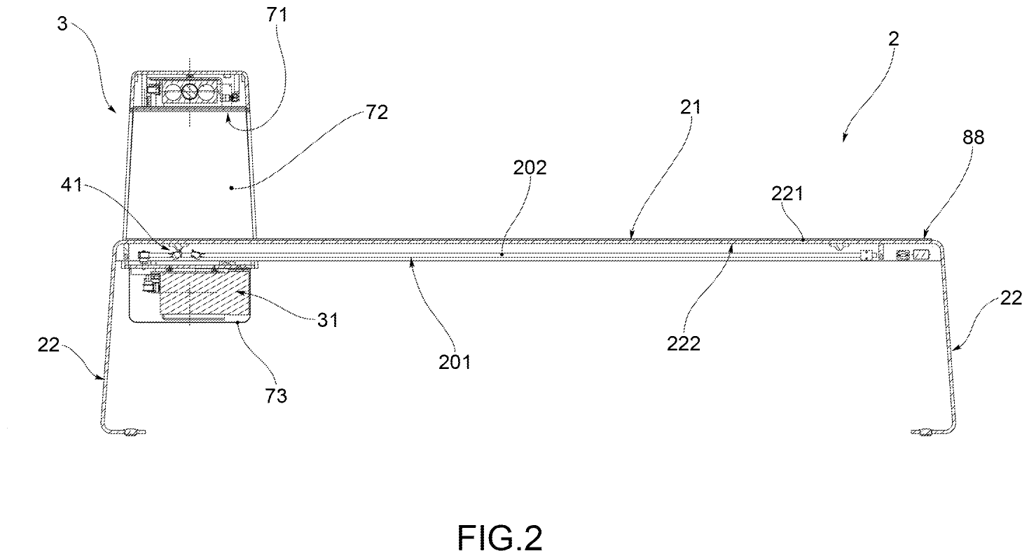

FIG. 2 is a front view of the apparatus in FIG. 1;

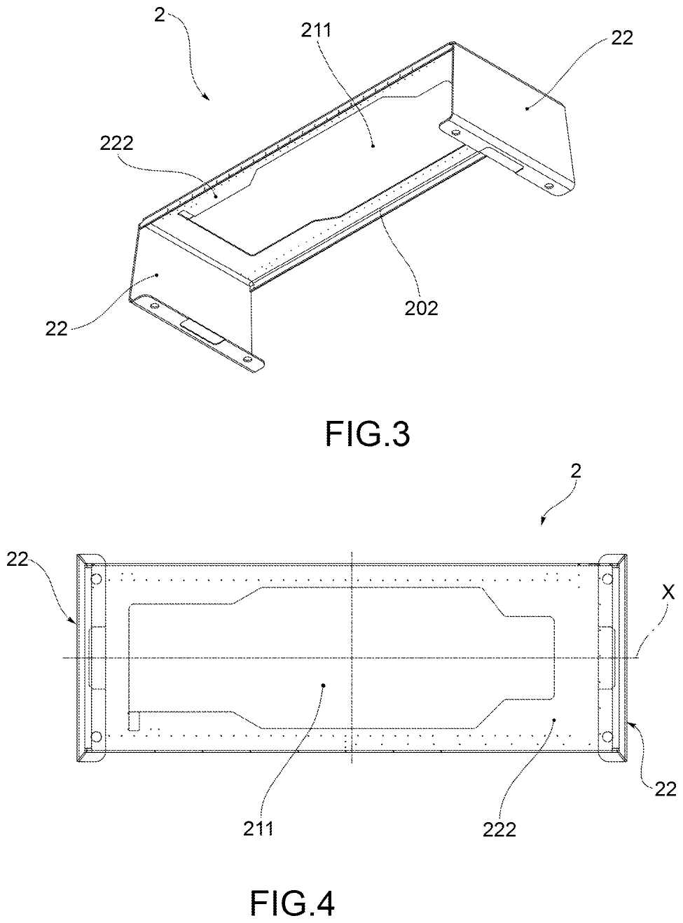

FIGS. 3 and 4 show both the lower portion of the patient-carrier frame in FIG. 1, in an axonometric view and a bottom view, respectively;

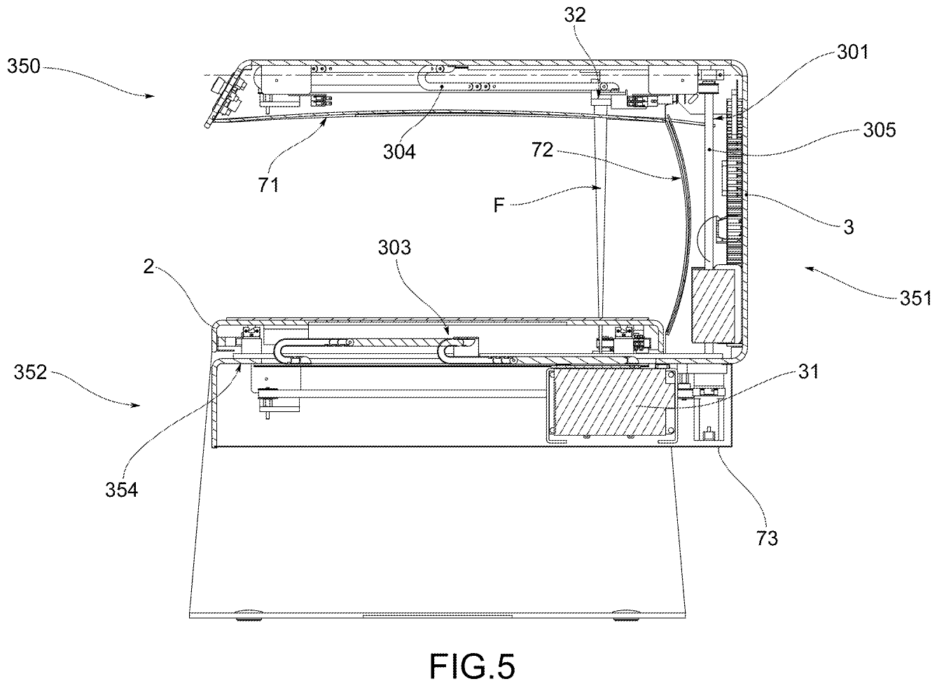

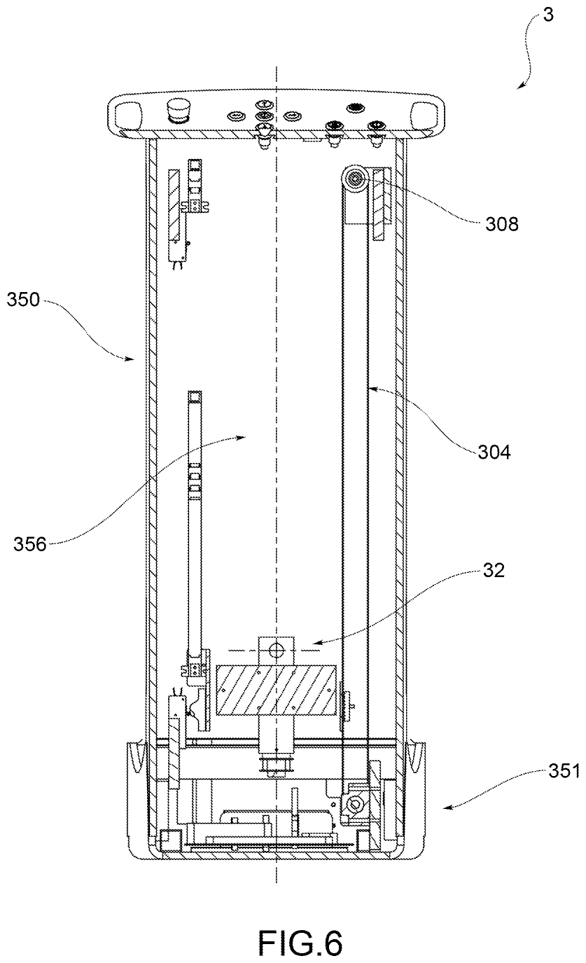

FIGS. 5 and 6 show both a section of the movable arm in FIG. 1, in a side view and a bottom view, respectively;

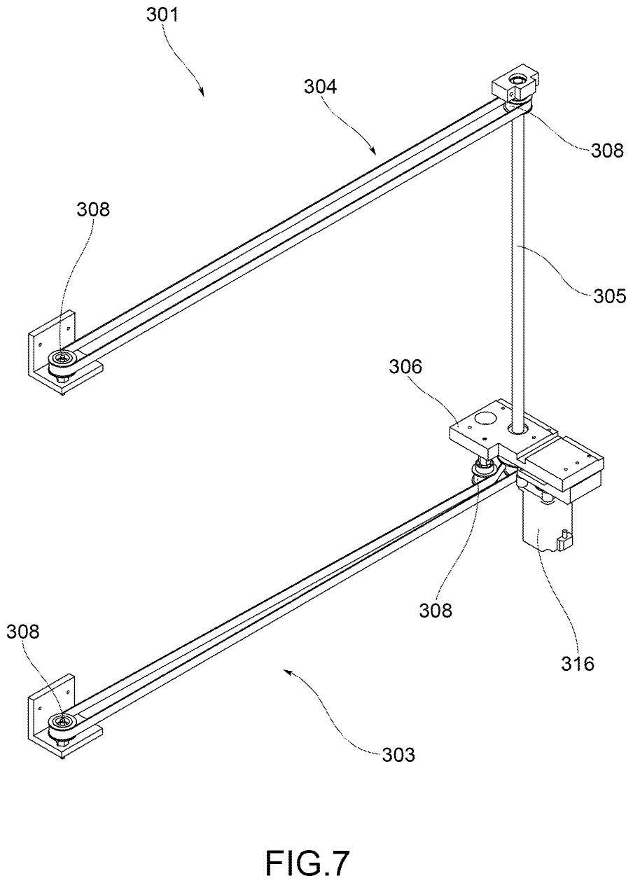

FIG. 7 shows a detail of the movement system of the movable arm;

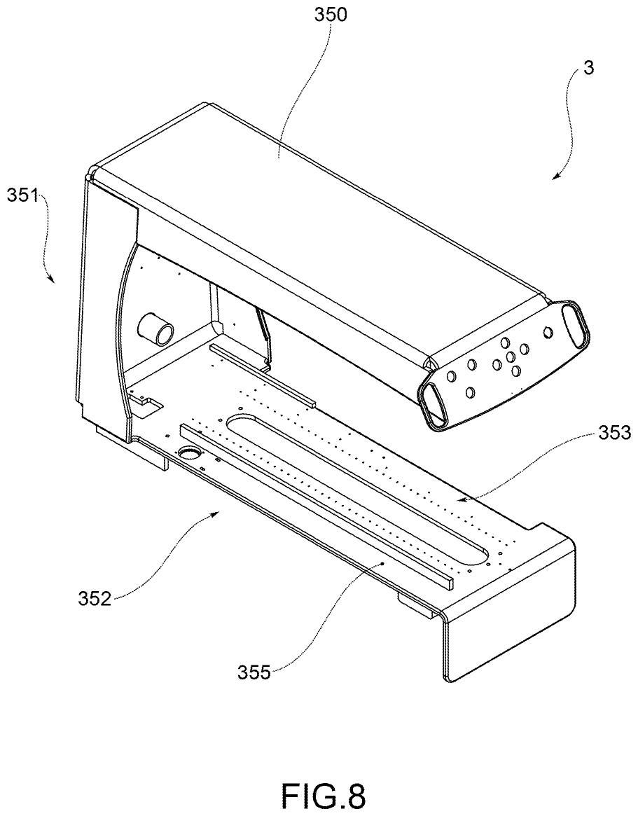

FIG. 8 shows the outer shell of the movable arm;

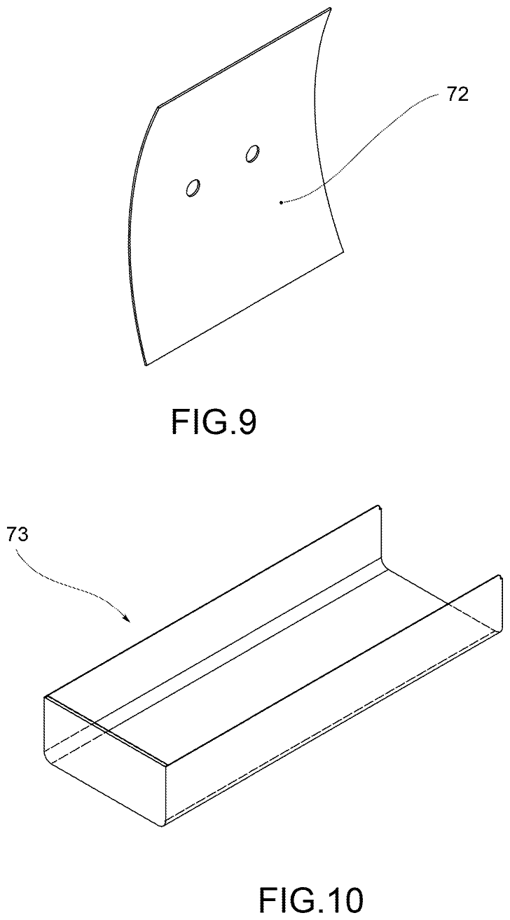

FIGS. 9 and 10 show further elements of completion, or casing, of the outer shell of the movable arm, respectively a front casing and a lower casing.

The apparatus for bone densitometry, indicated in the above figures with reference numeral 1, consists of mechanical, electronic and software parts integrated according to the technical regulations and to legislation in the field of medical devices.

The apparatus for bone densitometry 1 of the present invention uses a DEXA (Dual Energy X-Ray Absorptiometry) method.

As shown in FIG. 1, the apparatus for bone densitometry 1 comprises a patient-carrier frame 2 which supports an arm 3 movable along frame 2 and provided with a generator-detector assembly.

As shown also in FIG. 3, the apparatus for bone densitometry 1 is characterised by the presence of a monocoque patient-carrier frame 2 adapted to completely support, at the patient's support surface (i.e. the horizontal plane) the movable arm 3 fitted with the generator-detector assembly. Therefore, frame 2 acts both as a support for the patient and for arm 3.

The term “monocoque” means a single rigid structure with bearing function.

Therefore, in the apparatus for bone densitometry 1, the movable arm 3 is supported directly by frame 1 at the support surface 21 on which the patient lies. Such a solution allows having a completely free space below frame 2.

Advantageously, such a solution simplifies the cleaning of the area below the patient-carrier table or frame 2, thus improving the hygiene in the room where the apparatus for bone densitometry 1 is installed.

Advantageously, moreover, such a solution greatly reduces the number of the constituent parts of the apparatus for bone densitometry 1, almost completely eliminating the need for covers. Moreover, such a solution allows achieving a significant ease of transport and installation, as well as easier access for maintenance.

The apparatus for bone densitometry 1 acts as a planar scanner, being formed by a fixed stand (patient-carrier frame 2) and by an arm 3 which moves along the longitudinal direction X, from the feet to the head of the patient.

Arm 3 is fitted with a generator-detector assembly, mechanically aligned so that the collimated beam F of X-rays produced by generator 31 perfectly centres detector 32.

Generator 31 and detector 32 integrally translate both in the longitudinal direction X, by means of a longitudinal movement system 201, and in a transverse direction Y, by means of a transverse movement system 301. During the movement along axis Y, one or more lines of an image are acquired by attenuation of X-rays, while the translation along axis X serves for positioning at the next line. Through the longitudinal and transverse movement of the generator-detector assembly, an X-Y scan of a certain region of interest of the patient is achieved.

The apparatus for bone densitometry 1 further comprises a control unit, which implements the acquisition of the signals coming from detector 32, the control of x-ray emission from generator 31, and the control of the generator-detector assembly movement. The control unit also comprises software for bone densitometry.

An exemplary embodiment of the apparatus for bone densitometry 1 will now be described in detail. As shown in FIG. 2, frame 2 comprises a support surface 21, on which the patient lies, supported by legs 22.

The support surface 21 comprises an upper side 221 on which the patient lies, and a lower side 222, opposite the upper side 221, to which the longitudinal movement system 201 of arm 3 is attached.

In a preferred example, frame 2 is made starting from a single sheet of metal, folded to form a patient-carrier table.

In particular, frame 2 consists of a single sheet of folded aluminium. In particular, frame 2 consists of a single sheet of aluminium having a thickness of 10 mm. Advantageously, such a solution ensures structural strength: in fact, frame 2 allows a deformation not exceeding 1÷2 mm when loaded with a weight of 100 kg at the centre of the support surface 21.

Preferably, the support surface 21 of the patient comprises a scanning area 211 at which the patient lies. Preferably, the scanning area 211 has a length of about 200 cm and a width of about 70 cm.

The scanning area 211 is obtained by reducing the thickness of frame 2 (for example, originally 10 mm) to 4 mm. Such a solution allows carrying out a filtration of the X-ray beam necessary to eliminate the lower energy component. Frame 2 then comprises a filtering portion for X-rays, formed on the scanning area 211.

The apparatus for bone densitometry 1 is therefore also characterised in that frame 2 itself acts as a filter for the X-rays

In a further example, frame 2 is made starting from a single sheet of carbon fibre, folded to form a patient-carrier table.

The dimensions of frame 2 are such as to enable scanning the whole body of the patient, even for patients with high height and high weight.

For example, the support surface 21 has a length of about 2.5 metres and a width of about 1 metre.

Preferably, in order to facilitate the positioning of the patient on frame 2, the support surface 21 is placed at a height of about 60 cm from the floor surface.

The longitudinal movement system 201 of arm 3 is mounted on frame 2, at the lower side 222 of the support surface 21.

Preferably, as shown in FIG. 8, arm 3 is also monocoque.

In a preferred exemplary embodiment, arm 3 is made starting by folding a single sheet of metal, preferably aluminium with a thickness of 10 mm. Advantageously, arm 3 is considerably light, for example not exceeding 40 kg in weight.

Preferably, arm 3 is “U”-shaped.

As shown in FIG. 5, arm 3 comprises an upper portion 350, an intermediate portion 351, and a lower portion 352.

Arm 3 comprises all the active parts of the machine: X-ray generator 31 and X-ray detector 32, a pointer for the positioning of the patient, control electronics, motors 306, 316 for the longitudinal and transverse movements of arm 3, as well as, for example, a keyboard 310 for movement, a button 311 for emergency stop, buttons for movement and laser starting and signalling LEDs.

Preferably, arm 3 comprises, at the upper portion 350, a support plate 356 for detector 32 and for the pointer. The pointer is a low-power semiconductor laser, which acts as a reference point for the purposes of patient centring.

Preferably, arm 3 comprises, at the upper portion 350: detector 32, a pointer, keyboard 310, emergency button 311, buttons for movement and laser starting and signalling LEDs.

Preferably, arm 3 comprises, at the central portion 351: control and communication electronics, power supplies, motor drivers.

Preferably, arm 3 comprises, at the lower portion 352: generator 31, motors 306, 316 for the longitudinal and transverse movements.

Preferably, arm 3 comprises, at the lower portion 352, a support plate 353 for generator 31. In particular, generator 3 is hung on the lower side 354 of the support plate 353. The transverse movement system 301, and in particular the runners of guide 303 for moving generator 31, is instead attached at the upper side 355 of the support plate 353.

As mentioned above, the generator-detector assembly translate both in the longitudinal direction X, by means of the longitudinal movement system 201, and in a transverse direction Y, by means of the transverse movement system 301. The transmission of both the longitudinal (axis X) and transverse (axis Y) movement consists of a set of linear guides, carriages, belts, pulleys and bearings. Both motor 306 for the transverse movement and motor 316 for the longitudinal movement are mounted on arm 3.

The whole arm 3 is movable longitudinally along the patient-carrier frame 1 (i.e. along axis X) by means of the longitudinal movement system 201 supported directly by frame 1.

The longitudinal movement (axis X) takes place by dragging over a fixed toothed belt. Motor 316 transmits the movement through a system of belts and pulleys.

The longitudinal movement system 201 comprises a toothed belt drive, coupled to a pair of guides 202 mounted on the lower side 222 of the support surface 21. In this way, arm 3 moves suspended hung on frame 21, leaving completely free access to the underlying floor.

The linear guides 202 fitted with suitable carriages, support arm 3 and allow the translation thereof along axis X.

The mechanical connection between arm 3 and frame 2 is achieved by means of carriages 41.

The transverse movement system 301 of the generator-detector assembly is mounted on arm 3. Once the alignment has been adjusted, generator 31 and detector 32 move integrally and maintain the alignment along axis Y.

The movements of detector 32 and generator 31 are made integral precisely through the transverse movement system, in such a way that generator 31 and detector 32 achieve a perfect mechanical alignment with respect to the direction of the ray beam F.

As shown in FIG. 7, the transverse movement system 301 comprises a belt 303 for the movement of generator 31, placed in the lower part of arm 3, and a belt 304 for the movement of detector 32, placed in the upper part of arm 3.

Each belt 303, 304, preferably toothed, is wound around a respective pair of pulleys 308.

The transverse movement system 301 further comprises a drive rod 305, or a drive belt, for the transmission of motion from motor 306 to belts 303, 304, and for the correct synchronisation of the movement of generator 31 of detector 32.

The apparatus for bone densitometry 1 is also characterised in that the cover elements (also called casing) of the active parts of the machine housed in arm 3 are reduced to the minimum.

In particular, the cover elements comprise an upper casing 71, a central casing 72, and a lower casing 73.

The upper casing 71 is placed to close the upper portion 350 of arm 3 and to cover detector 32 by the whole stroke of movement Y. The upper casing 71 is preferably made in the form of a sheet of Plexiglas and is inserted into dedicated guides provided in the upper portion 350.

The central casing 72 is placed to close the central portion 351 and to cover the electronics mounted on arm 3. The central casing 72 is preferably made of aluminium.

The lower casing 73 is placed to close the lower portion 352 and to cover generator 31 and guide 303.

Preferably, the apparatus for bone densitometry 1 further comprises accessories which allow the patient to be subjected to the examination in comfortable conditions.

Preferably, the support surface 21 on which the patient lies is covered with a mat 88 of X-ray transparent material.

Preferably, the surface of mat 88 is washable and waterproof, in order to limit the risk of microbial contamination due to the promiscuity of use with potentially infected patients.

Preferably, mat 88 is made with a made fireproof and anti-allergic material.

Preferably, a brass collimator is mounted on the outlet mouth of generator 31. The collimator is thick enough to ensure the total shielding of the rays outside of the collimated beam (at least 20 mm). The geometry of the collimator ensures, in addition to the collimation, also shielding from X-rays outside the primary beam.

Preferably, a brass collimator (10 mm) diameter is also mounted on the detector 32 side. The collimation on the detector side serves to minimise the effect of any scattered radiation; it constitutes a means to adjust the efficiency of the detector and the system spatial resolution.

Advantageously, all the constituent elements of the apparatus for bone densitometry 1 are made in one piece, possibly folded, without the need for screws: monocoque frame 2, monobloc arm 3, casings 71, 72, 73.

Innovatively, an apparatus for bone densitometry according to the present invention has a very simple structure that allows having free space below the patient-carrier table, while being able to withstand high weight and height patients.

To summarise, therefore, the innovative and advantageous aspects of the apparatus for bone densitometry 1 according to the present invention:
monocoque patient-carrier frame 2 adapted to support, at the support surface 21 for the patient, the movable arm 3 fitted with a generator-detector assembly: area under the table completely clear and easy to clean; easy accessibility for patients with locomotory problems (e.g. wheelchair) who can be positioned close to the table, getting on and off independently;
apparatus for bone densitometry 1 consisting of an extremely small number of components: monobloc frame 2, monobloc arm 3, only three casings 71, 72, 73: ease of transport, mounting simplicity, rapidity in the possible need to move, installation and maintenance;
patient-carrier frame 2 fitted with a filtering portion for X-rays, consisting of the scanning area 211: possibility of making a metal frame (aluminium alloy) capable of supporting patients of considerable height and weight (even up to 410 kg and 2 m).

A man skilled in the art may make several changes and adjustments to the apparatus for bone densitometry described above in order to meet specific and incidental needs, all falling within the scope of protection defined in the following claims.

1.-13. (canceled)

14. An apparatus for bone densitometry, comprising:
a patient-carrier frame with a support surface for a patient;
an arm movable with respect to the frame in a longitudinal direction, from the feet to the head of the patient, and fitted with a generator-detector assembly comprising an X-ray generator and an X-ray detector, mechanically aligned so that a collimated beam of X-rays produced by the generator centers the detector;
wherein the frame is a monocoque and fully supports, at the support surface, the arm in order to leave a space underneath the frame free.

15. The apparatus for bone densitometry according to claim 14, wherein the support surface comprises an upper side on which the patient lies, and a lower side, opposite the upper side, wherein a longitudinal movement system of the arm is attached to the lower side.

16. The apparatus for bone densitometry according claim 14, wherein the frame comprises, at the support surface, a filtering portion for the X-rays.

17. The apparatus for bone densitometry according to claim 14, wherein the frame is made from a single sheet of aluminum folded to form a patient-carrier table.

18. The apparatus for bone densitometry according to claim 17, wherein the support surface comprises a scanning area in correspondence with which the patient lies down, wherein the scanning area comprises a reduced thickness of the frame to create a filtering portion for the X-rays.

19. The apparatus for bone densitometry according to claim 18, wherein the frame is made starting from a single sheet of aluminum having a thickness of 10 mm and in correspondence with the scanning area the thickness of the frame is reduced to 4 mm.

20. The apparatus for bone densitometry according to claim 14, wherein the frame is made from a single sheet of carbon fiber folded to form a patient-carrier table.

21. The apparatus for bone densitometry according to claim 14, wherein the generator-detector assembly translates with respect to the frame both in the longitudinal direction, by a longitudinal movement system, and in a transverse direction, by a transverse movement system so that a scan of a given region of interest of the patient is performed.

22. The apparatus for bone densitometry according to claim 21, wherein the longitudinal movement system comprises a toothed belt drive, coupled to a pair of guides mounted on the lower side of the support plane.

23. The apparatus for bone densitometry according to claim 21, wherein the transverse movement system comprises a belt for movement of the generator, placed in a lower part of the arm, and a belt for movement of the detector, placed in an upper part of the arm, connected by a drive belt for transmission of the movement from a motor to the belts and for the synchronization of the movement of the generator and of the detector.

24. The apparatus for bone densitometry according to claim 14, wherein the arm is monocoque and is fitted with cover elements of active parts of the machine.

25. The apparatus for bone densitometry according to claim 14, consisting of two elements: the frame and the arm.

26. The apparatus for bone densitometry according to claim 14, comprising a control unit suitable to implement Dual Energy X-ray Absorptiometry (DEXA) technology.
